(19) United States
(12) Patent Application Publication
(10) Pub. No.: US 2024/0122645 A1
(43) Pub. Date: Apr. 18, 2024

(54) MEDICAL DEVICE AND METHOD FOR FORMING SHUNT

(71) Applicant: TERUMO KABUSHIKI KAISHA, Tokyo (JP)

(72) Inventor: Yusuke TAKAHASHI, Hadano-shi, Kanagawa (JP)

(73) Assignee: TERUMO KABUSHIKI KAISHA, Shibuya-ku, Tokyo (JP)

(21) Appl. No.: 18/398,703

(22) Filed: Dec. 28, 2023

Related U.S. Application Data
(63) Continuation of application No. PCT/JP2022/016150, filed on Mar. 30, 2022.

(30) Foreign Application Priority Data
Jul. 9, 2021 (JP) 2021-114257

(51) Int. Cl. A61B 18/14

(52) U.S. Cl. CPC A61B 18/1492; A61B 2017/00252

(57) ABSTRACT

An expansion body, an elongated shaft portion to which the proximal end of the expansion body is fixed, a plurality of energy transfer elements disposed along the expansion body, and a pulling shaft are included, the pulling shaft is configured to apply, to the expansion body via a force receiving portion, a compressive force that makes compression along an axial center of the shaft portion such that a plurality of energy transfer element arrangement portions and a plurality of facing portions approach each other by sliding in a direction of the proximal end with respect to the shaft portion, and the expansion body includes a buffer portion that is disposed in a first expansion portion and is configured to relax the compressive force by deforming in a direction different from the direction from the force receiving portion toward a distal-side top portion along a distal-side expansion portion.

### CROSS-REFERENCES TO RELATED APPLICATIONS

This application is a continuation of International Application No. PCT/JP2022/016150 filed on Mar. 30, 2022, which claims priority to Japanese Application No. 2021-114257 filed on Jul. 9, 2021, the entire content of both of which is incorporated herein by reference.

### TECHNOLOGICAL FIELD

The present disclosure generally relates to a medical device including an expansion body that expands in a living body and a method for forming a shunt.

### BACKGROUND DISCUSSION

Chronic heart failure is a known heart disease. Chronic heart failure is broadly classified into a systolic heart failure and a diastolic heart failure on the basis of a cardiac function index. In a patient suffering from the diastolic heart failure, myocardial hypertrophy appears and stiffness (hardness) increases, whereby blood pressure increases in a left atrium and a cardiac pumping function is deteriorated. As a result, the patient may show heart failure symptoms such as a pulmonary edema. In addition, there is also a heart disease in which, due to pulmonary hypertension or the like, blood pressure increases on a right atrium side and the cardiac pumping function is deteriorated to exhibit heart failure symptoms.

In recent years, shunt treatments have attracted attention. For those patients who suffer from heart failure, a shunt (through-hole) serving as an escape route for increased atrial pressure is formed in an atrial septum, thereby helping alleviate heart failure symptoms. In the shunt treatment, the atrial septum is accessed using an intravenous approaching method, and the through-hole is formed to a desired size. Examples of a medical device for performing such a shunt treatment for the atrial septum include a device as disclosed in International Patent Application Publication No. WO 2020/094094 A.

In a medical device disclosed in International Patent Application Publication No. WO 2020/094094 A, a biological tissue is sandwiched by, from a distal side and a proximal side, two expandable expansion bodies around an axis of an elongated shaft, and electrode portions, which are a plurality of energy transfer elements arranged in a circumferential direction of one of the expansion bodies, are brought into contact with the biological tissue to be arranged in a circumferential direction of a hole of the biological tissue to be treated, and then energy is applied from the plurality of electrode portions to cauterize the biological tissue.

At a time of gripping the tissue to press the electrode against the tissue, a compressive force of the expansion body by a pulling shaft largely varies depending on thickness of the tissue. When the tissue is relatively thick, a compressive force larger than expected is applied to the electrode, whereby the hole of the biological tissue may be enlarged more than expected.

### SUMMARY

A medical device and a method for forming a shunt that are less likely to be affected by a variation in thickness of a biological tissue, suppress excessive expansion of a hole of the biological tissue, and enable highly safe and appropriate cauterization.

A medical device according to the present disclosure includes: an expansion body that includes a distal end part including a force receiving portion and is expandable/contractible in a radial direction; an elongated shaft portion including a distal end part to which a proximal end of the expansion body is fixed; a plurality of energy transfer elements disposed along the expansion body; and a pulling shaft that is disposed inside the shaft portion, connectable to the force receiving portion of the expansion body by protruding from the distal end part of the shaft portion, and slidable with respect to the shaft portion, in which the expansion body includes: a first expansion portion including a distal-side expansion portion extending radially outward from the force receiving portion toward a direction of the proximal end and a distal-side top portion disposed on a proximal side of the distal-side expansion portion and convexly curved radially outward; a second expansion portion including a proximal-side expansion portion extending radially outward from the distal end part of the shaft portion toward a direction of the distal end and a proximal-side top portion disposed on a distal side of the proximal-side expansion portion and convexly curved radially outward; and a recess that is recessed radially inward, extends to couple the proximal-side top portion with the distal-side top portion, and defines a reception space that can receive a biological tissue when the expansion body is expanded, the recess includes a bottom portion located on an innermost side in the radial direction, a distal-side upright portion extending radially outward from a distal end of the bottom portion to the distal-side top portion, and a proximal-side upright portion extending radially outward from a proximal end of the bottom portion to the proximal-side top portion, one of the distal-side upright portion or the proximal-side upright portion includes a plurality of energy transfer element arrangement portions on which the plurality of individual energy transfer elements is disposed at a substantially regular interval in a circumferential direction of the expansion body, the other one of the distal-side upright portion or the proximal-side upright portion includes a plurality of facing portions facing the plurality of individual energy transfer elements when the expansion body is expanded, the pulling shaft is configured to apply, to the expansion body via the force receiving portion, a compressive force that makes compression along an axial center of the shaft portion such that the plurality of energy transfer element arrangement portions and the plurality of facing portions approach each other by sliding in a direction of the proximal end with respect to the shaft portion, and the expansion body includes a buffer portion that is disposed in the first expansion portion and is configured to relax the compressive force by deforming in a direction different from a direction from the force receiving portion toward the distal-side top portion along the distal-side expansion portion, or a buffer portion that is disposed in the second expansion portion and is configured to relax the compressive force by deforming in a direction different from a direction from the proximal end of the expansion body toward the proximal-side top portion along the proximal-side expansion portion.

A method for forming a shunt according to the present disclosure forms, in an oval fossa, a shunt through which a right atrium communicates with a left atrium using a medical device including an expansion body that includes a distal end part including a force receiving portion and is expandable/contractible in a radial direction, an elongated shaft portion including a distal end part to which a proximal end of the expansion body is fixed, a plurality of energy transfer elements disposed along the expansion body, and a pulling shaft that is disposed inside the shaft portion, connectable to the force receiving portion of the expansion body by protruding from the distal end part of the shaft portion, and slidable with respect to the shaft portion, in which the expansion body includes a first expansion portion including a distal-side expansion portion extending radially outward from the force receiving portion toward a direction of the proximal end and a distal-side top portion disposed on a proximal side of the distal-side expansion portion and convexly curved radially outward, a second expansion portion including a proximal-side expansion portion extending radially outward from the distal end part of the shaft portion toward a direction of the distal end and a proximal-side top portion disposed on a distal side of the proximal-side expansion portion and convexly curved radially outward, and a recess that is recessed radially inward, extends to couple the proximal-side top portion with the distal-side top portion, and defines a reception space that can receive a biological tissue when the expansion body is expanded, the recess includes a bottom portion located on an innermost side in the radial direction, a distal-side upright portion extending radially outward from a distal end of the bottom portion to the distal-side top portion, and a proximal-side upright portion extending radially outward from a proximal end of the bottom portion to the proximal-side top portion, one of the distal-side upright portion or the proximal-side upright portion includes a plurality of energy transfer element arrangement portions on which the plurality of individual energy transfer elements is disposed at a substantially regular interval in a circumferential direction of the expansion body, the other one of the distal-side upright portion or the proximal-side upright portion includes a plurality of facing portions facing the plurality of individual energy transfer elements when the expansion body is expanded, the pulling shaft is configured to apply, to the expansion body via the force receiving portion, a compressive force that makes compression along an axial center of the shaft portion such that the plurality of energy transfer element arrangement portions and the plurality of facing portions approach each other by sliding in a direction of the proximal end with respect to the shaft portion, and the expansion body includes a buffer portion that is disposed in the first expansion portion and is configured to relax the compressive force by deforming in a direction different from a direction from the force receiving portion toward the distal-side top portion along the distal-side expansion portion, or a buffer portion that is disposed in the second expansion portion and is configured to relax the compressive force by deforming in a direction different from a direction from the proximal end of the expansion body toward the proximal-side top portion along the proximal-side expansion portion, the method including: inserting the medical device from an inferior vena cava into the right atrium; inserting the expansion body in a contracted state into a hole formed in the oval fossa; expanding the expansion body in the hole to dispose the biological tissue surrounding the hole in the reception space defined by the recess; sliding the pulling shaft in the direction of the proximal end with respect to the shaft portion to compress the expansion body such that the distal-side upright portion and the proximal-side upright portion of the recess approach each other; bringing the energy transfer elements disposed to face the recess along the distal-side upright portion or the proximal-side upright portion of the recess into contact with the biological tissue while relaxing the compressive force by deforming the buffer portion in a direction different from the direction from the force receiving portion toward the distal-side top portion along the distal-side expansion portion or relaxing the compressive force by deforming the buffer portion disposed in the second expansion portion in a direction different from the direction from the proximal end of the expansion body toward the proximal-side top portion along the proximal-side expansion portion; and cauterizing the biological tissue disposed in the reception space using the energy transfer elements in contact with the biological tissue to inhibit occlusion due to natural healing of the hole.

According to the medical device and the method for forming a shunt configured as described above, the compressive force is relaxed by deformation of the buffer portion in a direction different from the direction from the force receiving portion toward the distal-side top portion or in a direction different from the direction from the proximal end of the expansion body toward the proximal-side top portion when the compressive force is applied to the expansion body by pulling of the pulling shaft, whereby it becomes possible to suppress a state where the expansion body is excessively expanded radially outward. Therefore, it becomes possible to suppress variations in the amount of expansion of the expansion body toward the radial outside when the biological tissue received in the reception space is thick or thin. As a result, the present medical device and method for forming a shunt are less likely to be affected by variations in thickness of the biological tissue, suppress excessive expansion of the hole of the biological tissue, and enable highly safe and appropriate cauterization.

The first expansion portion may include a plurality of distal-side strut structures extending radially outward from the force receiving portion toward the direction of the proximal end and forming the distal-side expansion portion, and each of the plurality of distal-side strut structures may include, as the buffer portion, a bent portion bendable in a direction different from the direction from the force receiving portion toward the distal-side top portion along each of the distal-side strut structures. With this arrangement, the buffer portion may be implemented with a relatively simple structure.

Each of the plurality of distal-side strut structures may include a first section that includes a first strut extending from the force receiving portion substantially parallel to the axial center of the expansion body when viewed from the radial outside, and a second section that includes two second struts bifurcated from the proximal end of the first section substantially along the circumferential direction of the expansion body and is coupled to the distal-side top portion, and the second section may function as the buffer portion that relaxes the compressive force by bending such that a bifurcation angle formed by the two bifurcated second struts increases. With this arrangement, the second section of the distal-side strut structure is bent such that the bifurcation angle formed by the two bifurcated second struts increases, whereby the compressive force is relaxed and the state where the expansion body is excessively expanded radially outward may be suppressed.

The second section may include, in the vicinity of the distal-side top portion, a plurality of joint portions in which each of the two second struts joins one of the two second struts of another second section adjacent in the circumferential direction. With this arrangement, the adjacent distal-side strut structures support each other in the circumferential direction, whereby the expansion body is less likely to be twisted at the time of expansion.

The second section may include an auxiliary curved portion that functions as the buffer portion between the plurality of joint portions and the distal-side top portion disposed in the same phase as the energy transfer element arrangement portions or the facing portions in the circumferential direction of the expansion body. With this arrangement, the compressive force may be further relaxed by deformation of the auxiliary curved portion, whereby the compressive force is hardly converted into an expansive force.

The plurality of distal-side strut structures may include the first sections and the joint portions twice as many as the plurality of energy transfer elements, and the joint portions may alternately include, in the circumferential direction of the expansion body, a first joint portion disposed in the same phase as the plurality of energy transfer element arrangement portions and the plurality of facing portions in the circumferential direction of the expansion body, and a second joint portion disposed in a phase different from the phase of the plurality of energy transfer element arrangement portions and the plurality of facing portions. With this arrangement, the adjacent distal-side strut structures support each other in the circumferential direction, whereby the expansion body is less likely to be twisted at the time of expansion.

An auxiliary curved portion that functions as the buffer portion may be included between the first joint portion and the distal-side top portion. With this arrangement, the compressive force may be further relaxed by deformation of the auxiliary curved portion, whereby the compressive force is hardly converted into an expansive force.

The recess may include a recessed strut structure that is coupled to the distal-side strut structure via the distal-side top portion and defines the distal-side upright portion, the proximal-side upright portion, and the bottom portion, the recessed strut structure may include, in the bottom portion, a plurality of bottom connecting portions that couples individual pairs of the plurality of energy transfer element arrangement portions and the plurality of facing portions, and the plurality of bottom connecting portions may be disposed in a phase different from that of the first strut in the circumferential direction of the expansion body. With this arrangement, a portion extending in the circumferential direction is present between the first strut and the bottom connecting portion disposed in different phases, whereby a force is likely to act in a direction of widening the bifurcation angle, and the compressive force is less likely to be converted into the expansive force.

The energy transfer element arrangement portions may be disposed on the proximal-side upright portion, and the buffer portion may be disposed only on the distal-side expansion portion. With this arrangement, the energy transfer elements may be reliably pressed against the tissue when the expansion body is expanded.

The second expansion portion may include a plurality of proximal-side strut structures that extends radially outward from the distal end part of the shaft portion toward the direction of the distal end and forms the proximal-side expansion portion, and each of the plurality of proximal-side strut structures may include a third strut that is disposed in the same phase as the plurality of energy transfer element arrangement portions in the circumferential direction of the expansion body and extends from the distal end part of the shaft portion to the proximal-side top portion substantially parallel to the axial center of the expansion body when viewed from the radial outside. With this arrangement, the energy transfer elements may be reliably pressed against the tissue when the expansion body is expanded.

The second expansion portion may include a plurality of secondary struts that couples the third struts adjacent in the circumferential direction in the plurality of proximal-side strut structures, each of the plurality of secondary struts may include at least one support strut including two junctions joined to respective two third struts adjacent in the circumferential direction among a plurality of the third struts, and each of a plurality of the support struts may be formed to be longer than a linear distance between the two junctions. With this arrangement, the support strut may suppress a state where the proximal-side strut structure that has received the compressive force is twisted in the circumferential direction when the energy transfer elements are pressed against the tissue. Accordingly, in the medical device, the force of pressing the energy transfer elements against the tissue is less likely to be dispersed, whereby the energy transfer elements may be effectively pressed against the biological tissue.

The second expansion portion may include a plurality of proximal-side strut structures that extends radially outward from the distal end part of the shaft portion toward the direction of the distal end and forms the proximal-side expansion portion, each of the plurality of proximal-side strut structures may include a third section that includes a third strut extending from the distal end part of the shaft portion substantially parallel to the axial center of the expansion body when viewed from the radial outside, and a fourth section that includes two fourth struts bifurcated from the distal end of the third section substantially along the circumferential direction of the expansion body and is coupled to the proximal-side top portion, and the fourth section may function as the buffer portion that relaxes the compressive force by bending such that a bifurcation angle formed by the two bifurcated fourth struts increases. The fourth section of the proximal-side strut structure is bent such that the bifurcation angle formed by the two bifurcated fourth struts increases, whereby the compressive force is relaxed and the state where the expansion body is excessively expanded radially outward may be suppressed.

The fourth section may include, in the vicinity of the proximal-side top portion, a plurality of joint portions in which each of the two fourth struts joins one of the two fourth struts of another fourth section adjacent in the circumferential direction. With this arrangement, the adjacent proximal-side strut structures support each other in the circumferential direction, whereby the expansion body is less likely to be twisted at the time of expansion. Accordingly, an appropriate position of the biological tissue may be appropriately expanded by the expansion body, which enables appropriate cauterization.

An expansion body configured to be expandable and contractible in a radial direction according to the present disclosure includes: a distal end part including a force receiving portion; a first expansion portion including a distal-side expansion portion extending radially outward from the force receiving portion toward a direction of the proximal end and a distal-side top portion disposed on a proximal side of the distal-side expansion portion and convexly curved radially outward; a second expansion portion including a proximal-side expansion portion extending radially outward from the distal end part of the shaft portion toward a direction of the distal end and a proximal-side top portion disposed on a distal side of the proximal-side expansion portion and convexly curved radially outward; a recess that is recessed radially inward, extends to couple the proximal-side top portion with the distal-side top portion, and configured to define a reception space configured to receive a biological tissue when the expansion body is expanded; the recess includes a bottom portion located on an innermost side in the radial direction, a distal-side upright portion extending radially outward from a distal end of the bottom portion to the distal-side top portion, and a proximal-side upright portion extending radially outward from a proximal end of the bottom portion to the proximal-side top portion; one of the distal-side upright portion or the proximal-side upright portion includes a plurality of energy transfer element arrangement portions on which the plurality of individual energy transfer elements is disposed at a substantially regular interval in a circumferential direction of the expansion body; another one of the distal-side upright portion or the proximal-side upright portion includes a plurality of facing portions facing the plurality of individual energy transfer elements when the expansion body is expanded; and a buffer portion that is disposed in the first expansion portion and is configured to relax a compressive force by deforming in a direction different from a direction from the force receiving portion toward the distal-side top portion along the distal-side expansion portion, or a buffer portion that is disposed in the second expansion portion and is configured to relax the compressive force by deforming in a direction different from a direction from the proximal end of the expansion body toward the proximal-side top portion along the proximal-side expansion portion.

### BRIEF DESCRIPTION OF THE DRAWINGS

FIG. 1 is a side view illustrating an overall configuration of a medical device according to the present embodiment.

FIG. 2 is an enlarged perspective view of a vicinity of an expansion body of the medical device.

FIG. 3 is a side view illustrating a distal end part of the medical device.

FIG. 4 is a front view of the medical device as viewed from a distal side.

FIG. 5 is a schematic view schematically illustrating a state in which the expansion body is disposed in a through-hole of an atrial septum.

FIG. 6 is a cross-sectional view illustrating a state in which a balloon is inserted into the atrial septum.

FIG. 7 is a cross-sectional view illustrating a state in which the distal end part of the medical device is inserted into the atrial septum.

FIG. 8 is a cross-sectional view illustrating a state in which the expansion body is disposed in the atrial septum.

FIG. 9 is a cross-sectional view illustrating a state in which a plurality of energy transfer elements disposed in a recess of the expansion body is brought into contact with a biological tissue.

FIG. 10 is a flowchart for explaining a method for forming a shunt.

FIG. 11 is a side view illustrating an expansion body according to a first modified example.

FIG. 12 is a front view illustrating the expansion body according to the first modified example.

FIG. 13 is a side view illustrating an expansion body according to a second modified example.

FIG. 14 is a front view illustrating the expansion body according to the second modified example.

FIG. 15 is a side view illustrating an expansion body according to a third modified example.

FIG. 16 is a front view illustrating the expansion body according to the third modified example.

FIG. 17 is a side view illustrating an expansion body according to a fourth modified example.

FIG. 18 is a side view illustrating a state in which a compressive force in an axial direction is applied to the expansion body according to the fourth modified example.

FIG. 19 is a side view illustrating an expansion body according to a fifth modified example.

FIG. 20 is a side view illustrating an expansion body according to a sixth modified example.

### DETAILED DESCRIPTION

Set forth below with reference to the accompanying drawings is a detailed description of embodiments of a medical device including an expansion body that expands in a living body and a method for forming a shunt. Note that dimensional ratios in the drawings may be exaggerated and different from actual ratios for convenience of description. In addition, in the present specification, a side of a medical device to be inserted into a living body lumen is referred to as a “distal side” and a side to be operated is referred to as a “proximal side”.

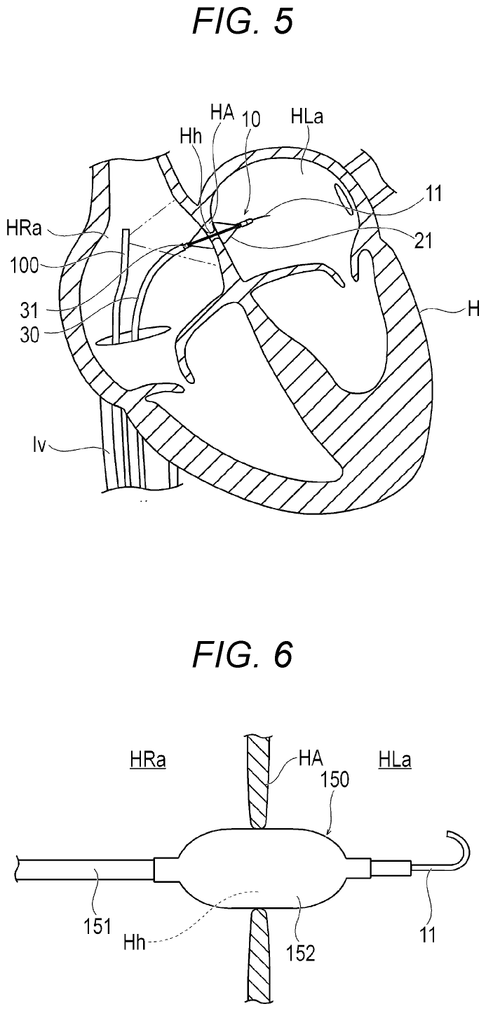

As illustrated in FIG. 5, a medical device 10 according to the present embodiment is configured to enlarge a through-hole Hh formed in an atrial septum HA of a heart H of a patient, and to further perform a maintenance treatment for maintaining the enlarged through-hole Hh at the increased size.

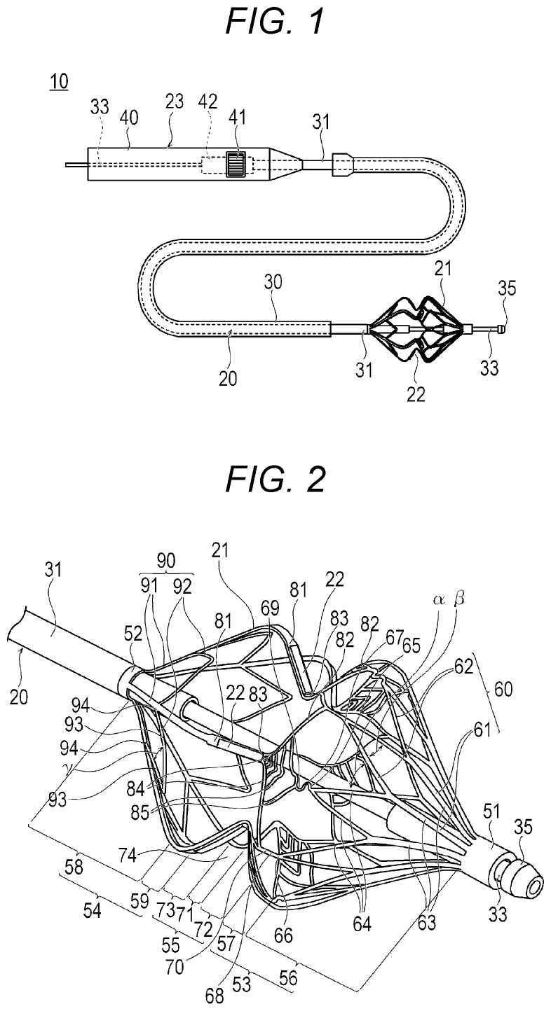

As illustrated in FIG. 1, the medical device 10 according to the present embodiment includes an elongated member 20 extending from a proximal end to a distal end, an expansion body 21 disposed on a distal end part of the elongated member 20, and an operation unit 23 connected to a proximal end part of the elongated member 20. An energy transfer element 22 (electrode portion) for performing the maintenance treatment described above is disposed on the expansion body 21.

As illustrated in FIGS. 1 to 3, the elongated member 20 includes a shaft portion 31 holding the expansion body 21 at a distal end part, an outer tube 30 that accommodates the shaft portion 31, a pulling shaft 33, and a pulling portion 35 fixed to the distal end of the pulling shaft 33.

The shaft portion 31 is an elongated tubular body extending from the operation unit 23 to the expansion body 21. A proximal end part of the shaft portion 31 is fixed to a distal end part of the operation unit 23. A distal end part of the shaft portion 31 is fixed to a proximal end part of the expansion body 21.

The outer tube 30 is an elongated tubular body covering the shaft portion 31, and is movable forward and backward with respect to the shaft portion 31 in the axial direction (direction of the axial center of the elongated member 20). The outer tube 30 is configured to accommodate the contracted expansion body 21 in the outer tube 30 in a state of being moved to the distal side of the elongated member 20. With the outer tube 30 being moved to the proximal side from the state of accommodating the expansion body 21, the expansion body 21 may be exposed.

The pulling shaft 33 is an elongated tubular body disposed inside the shaft portion 31, and is movable forward and backward with respect to the shaft portion 31 in the axial direction. The pulling shaft 33 protrudes from the distal end of the shaft portion 31 toward the distal side, and protrudes from the distal end of the expansion body 21 toward the distal side. A distal end part of the pulling shaft 33 on a side distal of the expansion body 21 is fixed to the pulling portion 35. A proximal end part of the pulling shaft 33 is drawn out (extends) to a side proximal of the operation unit 23. A guide wire lumen is formed in the pulling shaft 33 along the axial direction, and a guide wire 11 (see FIGS. 5 to 7) may be inserted into the guide wire lumen.

The pulling portion 35 is an annular member fixed to an outer peripheral surface of a distal end part of the pulling shaft 33, and protrudes radially outward from the outer peripheral surface of the pulling shaft 33. The pulling portion 35 is not fixed to the expansion body 21. The outer diameter of the pulling portion 35 is larger than the inner diameter of the distal end part of the expansion body 21. Therefore, the pulling portion 35 is enabled to abut on the distal end part of the expansion body 21 from the distal side, pull the expansion body 21 toward the direction of the proximal end, and apply a compressive force for making compression along the axial direction of the shaft portion 31 to the expansion body 21.

The operation unit 23 includes a housing 40 to be gripped by an operator, a dial 41 configured to be operated by the operator, and a conversion mechanism 42 that converts rotation of the dial 41 into movement in the axial direction. The dial 41 is rotatably coupled to the housing 40. The dial 41 is partially exposed to the outside from an opening of the housing 40 so as to be operated by the operator. The pulling shaft 33 is held by the conversion mechanism 42 inside the operation unit 23. The conversion mechanism 42 can move the holding pulling shaft 33 forward and backward along the axial direction in conjunction with the rotation of the dial 41. For example, a rack and pinion mechanism may be used as the conversion mechanism 42.

As illustrated in FIGS. 2 to 4, the expansion body 21 includes a force receiving portion 51 disposed at the distal end of the expansion body 21, a proximal-end connecting portion 52 disposed at the proximal end of the expansion body 21, a first expansion portion 53 coupled to the force receiving portion 51, a second expansion portion 54 coupled to the proximal-end connecting portion 52, and a recess 55 disposed between the first expansion portion 53 and the second expansion portion 54.

The force receiving portion 51 is annular, and is configured to receive a force directed toward the direction of the proximal end from the pulling portion 35 disposed on the distal side. The proximal-end connecting portion 52 is annular, and is fixed to the distal end part of the shaft portion 31.

The first expansion portion 53 includes a distal-side expansion portion 56 extending radially outward from the force receiving portion 51 toward the direction of the proximal end, and a distal-side top portion 57 disposed on the proximal side of the distal-side expansion portion 56 and convexly curved radially outward.

The first expansion portion 53 includes a plurality of distal-side strut structures 60 extending radially outward from the force receiving portion 51 toward the direction of the proximal end and forming the distal-side expansion portion 56.

Each of the plurality of distal-side strut structures 60 includes a first section 61 extending from the force receiving portion 51 toward the direction of the proximal end, and a second section 62 extending from the proximal end of the first section 61 toward the direction of the proximal end and coupled to the distal-side top portion 57.

Each of the first sections 61 includes a first strut 63 extending from the force receiving portion 51 substantially parallel to the axial center of the expansion body 21 when viewed from the radial outside.

Each of the second sections 62 includes a plurality of second struts 64 bifurcated to spread in the circumferential direction of the expansion body 21 while extending from the proximal end of each of the first struts 63 toward the direction of the proximal end, a first joint portion 65 and a second joint portion 66 coupled to the proximal end of the second strut 64, and an auxiliary curved portion 67 that functions as a buffer portion. The first joint portion 65 and the second joint portion 66 are alternately arranged at substantially regular intervals in the circumferential direction of the expansion body 21 at the time of expansion. Each of the first joint portion 65 and the second joint portion 66 is formed such that two second struts 64, which are bifurcated from respective two first struts 63 arranged on the distal side and adjacent in the circumferential direction and are extending to approach each other, are joined together. The number of the first struts 63 disposed in the expansion body 21 can be, for example, 12, which is twice the number of the energy transfer elements 22. The number of the second struts 64 disposed in the expansion body 21 can be, for example, 24, which is twice the number of the first struts 63 and four times the number of the energy transfer elements 22. Note that the number of the first struts 63 and the second struts 64 may be changed as appropriate.

Each of the first joint portions 65 is coupled to the distal-side top portion 57 disposed in the same phase as the energy transfer element 22 in the circumferential direction of the expansion body 21 via the auxiliary curved portion 67 that functions as a buffer portion. The auxiliary curved portion 67 is curved in a wavelike shape to be folded a plurality of times when viewed from the radial outside.

Each of the second joint portions 66 is coupled to the distal-side top portion 57 disposed in a different phase in the circumferential direction of the expansion body 21 with respect to the energy transfer element 22 with a connecting strut 68 extending substantially parallel to the axial center of the expansion body 21 interposed between the second joint portions 66 and the second distal-side top portion 70 when viewed from the radial outside.

The two second struts 64 bifurcated from the proximal ends of the respective first struts 63 are coupled at a first bifurcation angle α in a natural state. The two second struts 64 arranged in the circumferential direction are coupled to the first joint portion 65 or the second joint portion 66 at a second bifurcation angle β in the natural state.

The distal-side top portion 57 includes a plurality of first distal-side top portions 69 coupled to the auxiliary curved portion 67, and a plurality of second distal-side top portions 70 coupled to the connecting strut 68. The first distal-side top portions 69 and the second distal-side top portions 70 are alternately arranged at substantially regular intervals in the circumferential direction of the expansion body 21 at the time of expansion.

The recess 55 is recessed radially inward when the expansion body 21 is expanded, and extends to couple the proximal-side top portion 59 with the distal-side top portion 57. The recess 55 defines a reception space 74 configured to receive a biological tissue when the expansion body 21 is expanded.

The recess 55 includes the bottom portion 71 located on the innermost side in the radial direction, a distal-side upright portion 72 extending radially outward from the distal end of the bottom portion 71 to the distal-side top portion 57, and a proximal-side upright portion 73 extending radially outward from the proximal end of the bottom portion 71 to the proximal-side top portion 59.

The recess 55 includes a plurality of recessed strut structures 80 coupled to the plurality of distal-side strut structures 60 via the distal-side top portion 57. Each of the plurality of recessed strut structures 80 includes the energy transfer element arrangement portion 81 disposed on the proximal-side upright portion 73, and the facing portion 82 disposed on the distal-side upright portion 72, and also includes the bottom connecting portion 83 that couples a pair of the energy transfer element arrangement portion 81 and the facing portion 82 in the bottom portion 71. Each of the bottom connecting portions 83 is disposed in a phase different from that of the first strut 63 in the circumferential direction of the expansion body 21.

A plurality of the energy transfer element arrangement portions 81 is disposed at substantially regular intervals in the circumferential direction of the expansion body 21. The energy transfer element 22 is disposed on a surface of each of the energy transfer element arrangement portions 81 forming the inside of the recess 55.

The individual facing portions 82 face the individual energy transfer elements 22 when the expansion body 21 is expanded. Each of the facing portions 82 includes a plurality of distal-side upright struts 84 bifurcated to spread toward the direction of the distal end and a plurality of backrest portions 85 substantially along the circumferential direction of the expansion body 21 from the distal end of each of the bottom connecting portions 83. Each of the second distal-side top portions 70 is formed such that two distal-side upright struts 84, which are disposed on the proximal side and are extending to approach each other from the respective two bottom connecting portions 83 adjacent in the circumferential direction, are joined together. The plurality of backrest portions 85 couples the two distal-side upright struts 84 bifurcated from each of the bottom connecting portions 83. The plurality of backrest portions 85 is arranged side by side from the side closer to the bottom portion 71 to the side closer to the distal-side top portion 57. Each of the backrest portions 85 is curved such that a part between both ends coupled to the two distal-side upright struts 84 protrudes toward the distal-side top portion 57. Each of the backrest portions 85 is easily bent on the side closer to the distal-side top portion 57 with the both ends coupled to the distal-side upright struts 84 as supporting points. Therefore, the backrest portion 85 can be bent by a force toward the distal side received from the energy transfer element 22 disposed on the proximal-side upright portion 73. Accordingly, the biological tissue sandwiched between the energy transfer element 22 and the backrest portion 85 can be brought into contact with the energy transfer element 22. Among the plurality of backrest portions 85 forming each of the facing portions 82, the backrest portion 85 closest to the distal-side top portion 57 is coupled to the first distal-side top portion 69 at the part protruding toward the distal-side top portion 57. The number of the backrest portions 85 forming each of the facing portions 82 is not particularly limited.

The second expansion portion 54 includes a proximal-side expansion portion 58 extending radially outward from the proximal-end connecting portion 52 toward the direction of the distal end, and the proximal-side top portion 59 disposed on the distal side of the proximal-side expansion portion 58 and convexly curved radially outward.

The proximal-side expansion portion 58 includes a plurality of proximal-side strut structures 90. Each of the proximal-side strut structures 90 is disposed in the same phase as the plurality of energy transfer element arrangement portions 81 in the circumferential direction of the expansion body 21. Each of the plurality of proximal-side strut structures 90 includes a plurality of third struts 91 extending from the distal end part of the shaft portion 31 to the proximal-side top portion 59 substantially parallel to the axial center of the expansion body 21 when viewed from the radial outside, and a plurality of secondary struts 92 coupling the third struts 91 adjacent in the circumferential direction. Each of the secondary struts 92 includes two support struts 93 joined to, at a junction 94, individual two third struts 91 adjacent in the circumferential direction. The two support struts 93 are coupled to have an angle between two junctions 94. The coupled two support struts 93 are coupled at a coupling angle γ of less than 180 degrees in the natural state. Thus, each of the secondary struts 92 is formed to be longer than the linear distance between the two junctions 94. With this arrangement, even when the distance between the two junctions 94 increases at the time of expansion of the expansion body 21, the secondary strut 92 can continuously support the two third struts 91 while changing the coupling angle γ between the two support struts 93 included in the secondary strut 92. Therefore, the expansion body 21 is enabled to expand by the compressive force applied by the pulling shaft 33 while expanding the third struts 91 at substantially regular intervals.

An interval between the proximal-side upright portion 73 and the distal-side upright portion 72 is preferably slightly larger in the axial direction on the outer side than the inner side in the radial direction when the expansion portion is expanded. With this arrangement, the biological tissue can be easily arranged between the proximal-side upright portion 73 and the distal-side upright portion 72 from the radial outside.

The energy transfer element 22 is disposed on a surface toward the distal side of the proximal-side upright portion 73 when the expansion portion is expanded. Since the energy transfer element 22 is disposed on the proximal-side upright portion 73, energy from the energy transfer element 22 is transmitted to the atrial septum HA from the right atrium side when the recess 55 sandwiches the atrial septum HA. In a case where the energy transfer element 22 is disposed on the distal-side upright portion 72, the energy from the energy transfer element 22 is transmitted to the atrial septum HA from the left atrium side.

The energy transfer element 22 can include, for example, a bipolar electrode that receives electric energy from an energy supply device, which is an external device. In this case, electricity is conducted between the energy transfer elements 22 disposed on individual arrangement portions of the energy transfer elements 22. The energy transfer element 22 and the energy supply device are connected to each other by a conductive wire coated with an insulating coating material. The conductive wire is drawn out (extends) to the outside via the elongated member 20 and the operation unit 23, and is connected to the energy supply device.

Alternatively, the energy transfer element 22 may be configured as a monopolar electrode. In this case, electricity is supplied from a counter electrode plate prepared outside a body. Furthermore, the energy transfer element 22 may be a heating element (electrode chip) that receives high-frequency electric energy from the energy supply device and generates heat. In this case, electricity is conducted between the energy transfer elements 22 disposed on individual wire rod portions. Moreover, the energy transfer element 22 may include an element configured to apply energy to the through-hole Hh, such as a heater including an electric wire or the like that provides heating and cooling operation or generates frictional heat by using microwave energy, ultrasound energy, coherent light such as laser, a heated fluid, a cooled fluid, or a chemical medium, and a specific form is not particularly limited.

While the energy transfer element 22 and the backrest portion 85 are disposed on the proximal-side upright portion 73 and the distal-side upright portion 72, respectively, in the present embodiment, the energy transfer element 22 and the backrest portion 85 may be disposed on the distal-side upright portion 72 and the proximal-side upright portion 73, respectively.

The expansion body 21 is cut out from a cylinder to be integrally formed, for example. The struts forming the expansion body 21 may have a thickness, for example, in a range of 50 μm to 500 μm and a width, for example, in a range of 0.3 mm to 2.0 mm. However, the struts forming the expansion body 21 may have dimensions outside the ranges. In addition, a shape of the struts is not particularly limited, and may be, for example, a circular cross-sectional shape or another cross-sectional shape.

The expansion body 21 may be formed of a metal material. Examples of the metal material that may be used for the expansion body 21 can include a titanium-based (Ti—Ni, Ti—Pd, Ti—Nb—Sn, etc.) alloy, a copper-based alloy, stainless steel, p-titanium steel, and a Co—Cr alloy. Note that an alloy having a spring property, such as a nickel titanium alloy, or the like may be more preferably used. However, a material of the wire rod portions is not limited to the above materials, and the wire rod portions may be formed of other materials.

The outer tube 30 and the shaft portion 31 of the elongated member 20 are preferably formed of a material having a certain degree of flexibility. Examples of such a material for the outer tube 30 and the shaft portion 31 can include polyolefin such as polyethylene, polypropylene, polybutene, ethylene-propylene copolymer, ethylene-vinyl acetate copolymer, ionomer, or a mixture of two or more of the materials listed above, soft polyvinyl chloride resin, polyamide, polyamide elastomer, polyester, polyester elastomer, polyurethane, fluorine resin such as polytetrafluoroethylene, polyimide, polyetheretherketone (PEEK), silicone rubber, and latex rubber.

The pulling shaft 33 and the pulling portion 35 may be formed of, for example, an elongated wire rod including a super elasticity alloy such as a nickel-titanium alloy and a copper-zinc alloy, a metal material such as stainless steel, a resin material having comparatively high rigidity, or the like. Furthermore, the pulling shaft 33 and the pulling portion 35 may be formed of the materials described above coated with a resin material such as polyvinyl chloride, polyethylene, polypropylene, ethylene-propylene copolymer, fluorine resin, or the like.

Next, a method for forming a shunt using the medical device 10 according to the present embodiment will be described with reference to a flowchart illustrated in FIG. 10. The present method for forming a shunt is performed on a patient suffering from heart failure (left heart failure). More specifically, as illustrated in FIG. 5, the present method is a treatment method to be performed on a patient suffering from chronic heart failure in which myocardial hypertrophy appears in a left ventricle of the heart H and stiffness (hardness) increases so that blood pressure increases in a left atrium HLa.

The method for forming a shunt according to the present embodiment includes forming the through-hole Hh in the atrial septum HA (S1), disposing the expansion body 21 in the through-hole Hh (S2), receiving a biological tissue in the reception space 74 (S3), enlarging the diameter of the through-hole Hh using the expansion body 21 (S4), confirming hemodynamics in the vicinity of the through-hole Hh (S5), performing the maintenance treatment for maintaining the size of the through-hole Hh (S6), and confirming the hemodynamics in the vicinity of the through-hole Hh after the maintenance treatment (S7).

At the time of forming the through-hole Hh, the operator delivers, to the vicinity of the atrial septum HA, an introducer in which a guiding sheath and a dilator are combined with each other. For example, the introducer may be delivered to a right atrium HRa via an inferior vena cava Iv. In addition, the introducer may be delivered using the guide wire 11. The operator may insert the guide wire 11 into the dilator and deliver the introducer along the guide wire 11. The introducer and the guide wire 11 may be inserted into a living body using a method such as a method of using an introducer to be introduced into a blood vessel.

In the step of S1, the operator causes a puncture device to penetrate from the side of the right atrium HRa toward the side of the left atrium HLa to form the through-hole Hh in an oval fossa of the atrial septum HA. As the puncture device, a device such as a wire having a sharp distal end maybe used, for example. The puncture device is inserted into the dilator, and is delivered to the atrial septum HA. The puncture device may be delivered to the atrial septum HA instead of the guide wire 11 after the guide wire 11 is removed from the dilator.

Next, the operator delivers a balloon catheter 150 to the vicinity of the atrial septum HA along the guide wire 11 inserted in advance. As illustrated in FIG. 6, the balloon catheter 150 includes a balloon 152 at a distal end part of a shaft portion 151. When the balloon 152 is placed in the atrial septum HA, it is expanded in the radial direction to enlarge the through-hole Hh.

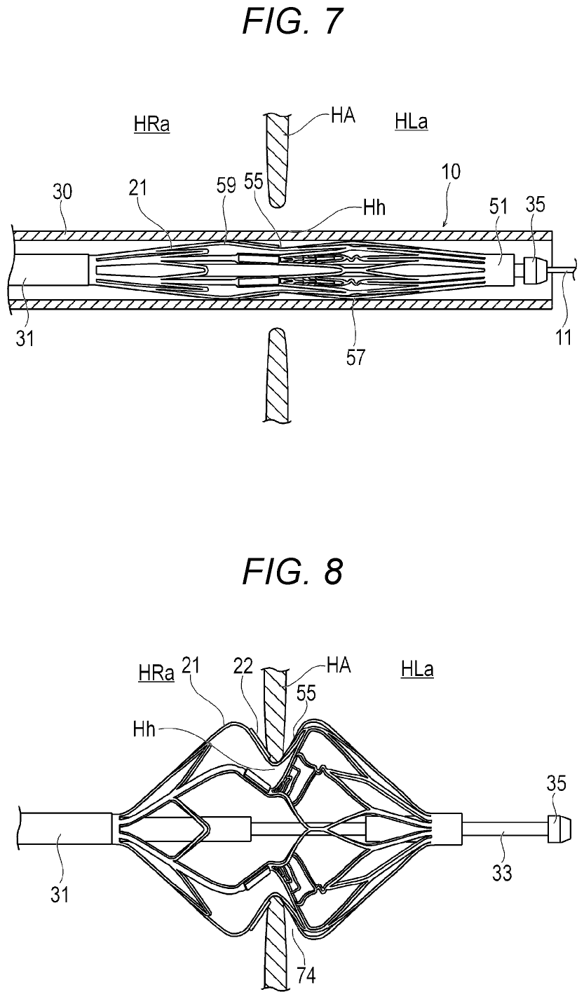

As illustrated in FIG. 7, in the step of S2, the medical device 10 is delivered to the vicinity of the atrial septum HA along the guide wire 11 inserted in advance. At this time, the distal end part of the medical device 10 penetrates the atrial septum HA and reaches the left atrium HLa. In addition, when the medical device 10 is inserted, the expansion body 21 is in a state of being housed in the outer tube 30.

Next, in the step of S3, the outer tube 30 is moved to the proximal side to expose the expansion body 21. As a result, as illustrated in FIG. 8, the diameter of the expansion body 21 increases and the recess 55 is arranged in the through-hole Hh of the atrial septum HA to receive the biological tissue surrounding the through-hole Hh in the reception space 74. The through-hole Hh is maintained in a state of being enlarged by the expansion body 21.

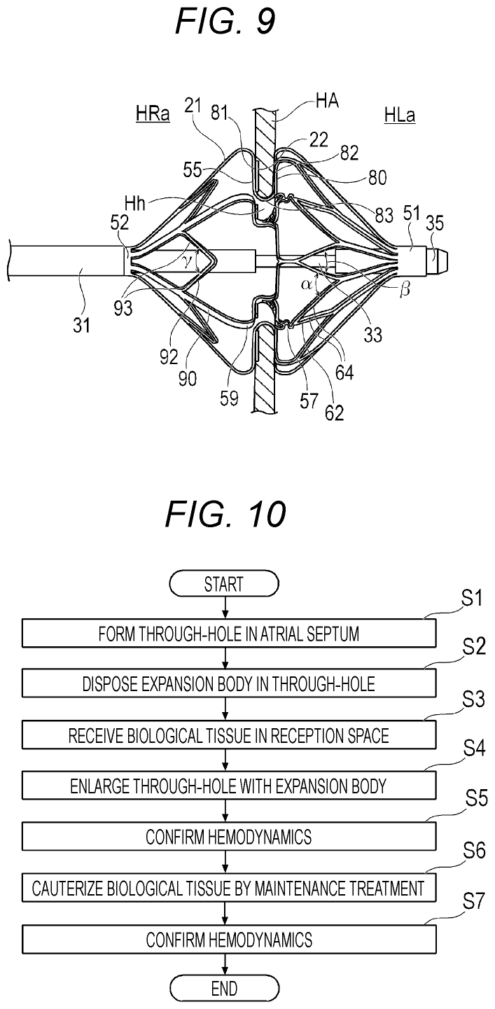

In the step of S4, the operator operates the operation unit 23 in the state where the atrial septum HA is received in the reception space 74 of the recess 55, moves the pulling shaft 33 to the proximal side, and sandwiches the biological tissue with the recess 55 of the expansion body 21, as illustrated in FIG. 9. Meanwhile, thickness of the atrial septum HA (biological tissue) varies among individuals, and may be thick or thin. When the atrial septum HA is thick, the second section 62 may be bent such that the first bifurcation angle α and the second bifurcation angle β of the second strut 64 increase when a compressive force is applied to the expansion body 21 by the pulling of the pulling shaft 33. That is, the second section 62, which serves as a buffer portion, is deformed in a direction different from the direction from the force receiving portion 51 toward the distal-side top portion 57. As a result, since the compressive force applied from the pulling shaft 33 is relaxed in the expansion body 21, it becomes possible to suppress a state where the expansion body 21 is excessively expanded radially outward. In addition, the auxiliary curved portion 67 between the distal-side top portion 57 and the first joint portion 65 is deformable in various directions. Thus, when the compressive force is applied to the expansion body 21 by the pulling of the pulling shaft 33, the auxiliary curved portion 67 also functions as a buffer portion that deforms in a direction different from the direction from the force receiving portion 51 toward the distal-side top portion 57, which may further relax the compressive force. In addition, when the compressive force is applied to the expansion body 21 by the pulling of the pulling shaft 33, the secondary strut 92 of the proximal-side strut structure 90 also deforms in a direction different from the direction from the proximal-end connecting portion 52 toward the proximal-side top portion 59. That is, the two support struts 93 included in each of the secondary struts 92 may be deformed while increasing the coupling angle γ to function as a buffer portion. Therefore, due to the deformation of each buffer portion, variations in the amount of expansion of the expansion body 21 toward the radial outside in the case of a thick biological tissue or a thin biological tissue may be suppressed. As a result, even when the thickness of the atrial septum HA is thick or thin, the through-hole Hh may have an appropriate size without being too large or too small.

After the expansion body 21 is disposed in the through-hole Hh, the hemodynamics is checked in the step of S5. As illustrated in FIG. 5, the operator delivers a hemodynamics checking device 100 to the right atrium HRa via the inferior vena cava Iv. For example, an echo catheter may be used as the hemodynamics checking device 100. The operator can display an echo image obtained by the hemodynamics checking device 100 on a display device, such as a display, and can check blood volume passing through the through-hole Hh on the basis of a displayed result.

Next, in the step of S6, the operator performs the maintenance treatment for maintaining the size of the through-hole Hh. In the maintenance treatment, high-frequency energy is applied to an edge portion of the through-hole Hh through the energy transfer element 22, thereby cauterizing (heating and cauterizing) the edge portion of the through-hole Hh with the high-frequency energy.

When the biological tissue in the vicinity of the edge portion of the through-hole Hh is cauterized through the energy transfer element 22, a degenerated portion in which the biological tissue is degenerated is formed in the vicinity of the edge portion. The biological tissue in the degenerated portion loses elasticity so that the through-hole Hh is enabled to maintain the shape enlarged by the expansion body 21. The through-hole Hh is held in an appropriate size by the expansion body 21 including the buffer portion and cauterized, which enables through-hole Hh to maintain the shape in the appropriate size.

The hemodynamics is checked again in the step of S7 after the maintenance treatment, and in a case where the blood volume passing through the through-hole Hh reaches desired volume, the operator decreases the diameter of the expansion body 21, stores the expansion body 21 in the outer tube 30, and removes the expansion body 21 from the through-hole Hh. Moreover, the operator removes the entire medical device 10 from the living body to the outside, and terminates the treatment.

As described above, the medical device 10 according to the present embodiment includes the expansion body 21 that includes a distal end part including the force receiving portion 51 and is expandable/contractible in the radial direction, the elongated shaft portion 31 including a distal end part to which the proximal end of the expansion body 21 is fixed, the plurality of energy transfer elements 22 (electrode portions) disposed along the expansion body 21, and the pulling shaft 33 that is disposed inside the shaft portion 31, connectable to the force receiving portion 51 of the expansion body 21 by protruding from the distal end part of the shaft portion 31, and slidable with respect to the shaft portion 31, in which the expansion body 21 includes the first expansion portion 53 including the distal-side expansion portion 56 extending radially outward from the force receiving portion 51 toward the direction of the proximal end and the distal-side top portion 57 disposed on the proximal side of the distal-side expansion portion 56 and convexly curved radially outward, the second expansion portion 54 including the proximal-side expansion portion 58 extending radially outward from the distal end part of the shaft portion 31 toward the direction of the distal end and the proximal-side top portion 59 disposed on the distal side of the proximal-side expansion portion 58 and convexly curved radially outward, and the recess 55 that is recessed radially inward, extends to couple the proximal-side top portion 59 with the distal-side top portion 57, and defines the reception space 74 that can receive the biological tissue when the expansion body 21 is expanded, the recess 55 includes the bottom portion 71 located on the innermost side in the radial direction, the distal-side upright portion 72 extending radially outward from the distal end of the bottom portion 71 to the distal-side top portion 57, and the proximal-side upright portion 73 extending radially outward from the proximal end of the bottom portion 71 to the proximal-side top portion 59, one of the distal-side upright portion 72 or the proximal-side upright portion 73 includes the plurality of energy transfer element arrangement portions 81 on which the plurality of individual energy transfer elements is disposed at substantially regular intervals in the circumferential direction of the expansion body 21, another one of the distal-side upright portion 72 or the proximal-side upright portion 73 includes the plurality of facing portions 82 facing the plurality of individual energy transfer elements when the expansion body 21 is expanded, the pulling shaft 33 is configured to apply, to the expansion body 21 via the force receiving portion 51, a compressive force that makes compression along the axial center of the shaft portion 31 such that the plurality of energy transfer element arrangement portions 81 and the plurality of facing portions 82 approach each other by sliding in the direction of the proximal end with respect to the shaft portion 31, and the expansion body 21 includes the buffer portion that is disposed in the first expansion portion 53 and is configured to relax the compressive force by deforming in a direction different from the direction from the force receiving portion 51 toward the distal-side top portion 57 along the distal-side expansion portion 56, or the buffer portion that is disposed in the second expansion portion 54 and is configured to relax the compressive force by deforming in a direction different from the direction from the proximal end of the expansion body 21 toward the proximal-side top portion 59 along the proximal-side expansion portion 58.

According to the medical device 10 configured as described above, the compressive force is relaxed by deformation of the buffer portion in a direction different from the direction from the force receiving portion 51 toward the distal-side top portion 57 or in a direction different from the direction from the proximal end of the expansion body 21 toward the proximal-side top portion 59 when the compressive force is applied to the expansion body 21 by the pulling of the pulling shaft 33, whereby it becomes possible to suppress the state where the expansion body 21 is excessively expanded radially outward. Therefore, it becomes possible to suppress variations in the amount of expansion of the expansion body 21 toward the radial outside when the biological tissue received in the reception space 74 of the expansion body 21 is thick or thin. Thus, the medical device 10 is enabled to suppress variations in the amount of radial expansion of the expansion body 21, which is inserted into the hole penetrating the biological tissue and is expanded radially outward by the compressive force, due to variations in the thickness of the biological tissue. Therefore, the present medical device 10 is less likely to be affected by the variations in the thickness of the biological tissue, suppresses excessive expansion of the hole of the biological tissue, and enables highly safe and appropriate cauterization.

Furthermore, the first expansion portion 53 includes the plurality of distal-side strut structures 60 extending radially outward from the force receiving portion 51 toward the direction of the proximal end and forming the distal-side expansion portion 56, and each of the distal-side strut structures 60 includes, as a buffer portion, the bent portion (second strut 64) bendable in a direction different from the direction from the force receiving portion 51 toward the distal-side top portion 57 along each of the distal-side strut structures 60. With this arrangement, the buffer portion may be implemented with a relatively simple structure.

Furthermore, each of the plurality of distal-side strut structures 60 includes the first section 61, which includes the first strut 63 extending from the force receiving portion 51 substantially parallel to the axial center of the expansion body 21 when viewed from the radial outside, and the second section 62, which includes the two second struts 64 bifurcated from the proximal end of the first section 61 substantially along the circumferential direction of the expansion body 21 and is coupled to the distal-side top portion 57, and the second section 62 functions as a buffer portion that relaxes the compressive force by being bent such that the first bifurcation angle α and the second bifurcation angle β formed by the two bifurcated second struts 64 increase. As a result, the second section 62 of the distal-side strut structure 60 is bent such that the first bifurcation angle α and the second bifurcation angle β formed by the two bifurcated second struts 64 increase, whereby the compressive force is relaxed and the state where the expansion body 21 is excessively expanded radially outward may be suppressed.

Furthermore, the second section 62 includes, in the vicinity of the distal-side top portion 57, a plurality of the first joint portions 65 and the second joint portions 66 in which each of the two second struts 64 joins one of the two second struts 64 of another second section 62 adjacent in the circumferential direction. As a result, the adjacent distal-side strut structures 60 support each other in the circumferential direction, whereby the expansion body 21 is less likely to be twisted at the time of expansion. Therefore, an appropriate position of the biological tissue may be appropriately expanded by the expansion body 21, which helps enable appropriate cauterization.

Furthermore, the second section 62 includes the auxiliary curved portion 67, which functions as a buffer portion, between the plurality of first joint portions 65 and the distal-side top portion 57 disposed in the same phase as the energy transfer element arrangement portion 81 or the facing portion 82 in the circumferential direction of the expansion body 21. As a result, the compressive force may be further relaxed by deformation of the auxiliary curved portion 67, whereby the compressive force is hardly converted into an expansive force.

Furthermore, the plurality of distal-side strut structures 60 includes the first sections 61 and the joint portions (first joint portion 65 and second joint portion 66), which are twice as many as the plurality of energy transfer elements 22 (electrode portions), and the joint portions alternately include the first joint portions 65 disposed in the same phase as the plurality of energy transfer element arrangement portions 81 and the plurality of facing portions 82 and the second joint portions 66 disposed in a phase different from that of the plurality of energy transfer element arrangement portions 81 and the plurality of facing portions 82 in the circumferential direction of the expansion body 21. As a result, the adjacent distal-side strut structures 60 support each other in the circumferential direction, whereby the expansion body 21 is less likely to be twisted at the time of expansion. Therefore, an appropriate position of the biological tissue may be appropriately expanded by the expansion body 21, which enables appropriate cauterization.

Furthermore, the medical device 10 includes the auxiliary curved portion 67 that functions as a buffer portion between the first joint portion 65 and the distal-side top portion 57. As a result, the compressive force may be further relaxed by deformation of the auxiliary curved portion 67, whereby the compressive force is hardly converted into an expansive force.

Furthermore, the recess 55 includes the recessed strut structure 80 that is coupled to the distal-side strut structure 60 via the distal-side top portion 57 and defines the distal-side upright portion 72, the proximal-side upright portion 73, and the bottom portion 71, the recessed strut structure 80 includes, in the bottom portion 71, the plurality of bottom connecting portions 83 that couples individual pairs of the plurality of energy transfer element arrangement portions 81 and the plurality of facing portions 82, and the plurality of bottom connecting portions 83 is disposed in a phase different from that of the first strut 63 in the circumferential direction of the expansion body 21. As a result, a portion extending in the circumferential direction is present between the first strut 63 and the bottom connecting portion 83 disposed in different phases, whereby a force is likely to act in a direction of widening the bifurcation angle, and the compressive force is less likely to be converted into the expansive force.

Furthermore, the energy transfer element arrangement portion 81 is disposed on the proximal-side upright portion 73, and the buffer portion is disposed only on the distal-side expansion portion 56. As a result, when the expansion body 21 is expanded, the second expansion portion 54 effectively transmits the compressive force so that the energy transfer elements may be reliably pressed against the tissue.

Furthermore, the second expansion portion 54 includes the plurality of proximal-side strut structures 90 extending radially outward from the distal end part of the shaft portion 31 toward the direction of the distal end and forming the proximal-side expansion portion 58, and each of the plurality of proximal-side strut structures 90 includes the third strut 91 disposed in the same phase as the plurality of energy transfer element arrangement portions 81 in the circumferential direction of the expansion body 21 and extending from the distal end part of the shaft portion 31 to the proximal-side top portion 59 substantially parallel to the axial center of the expansion body 21 when viewed from the radial outside. As a result, when the expansion body 21 is expanded, the second expansion portion 54 effectively transmits the compressive force so that the energy transfer elements may be reliably pressed against the tissue.

Furthermore, the second expansion portion 54 includes the plurality of secondary struts 92 that couples the third struts 91 adjacent in the circumferential direction in the plurality of proximal-side strut structures, each of the plurality of secondary struts 92 includes at least one support strut 93 having two junctions 94 joined to the respective two third struts 91 adjacent in the circumferential direction among the plurality of third struts 91, and each of a plurality of the support struts 93 is formed to be longer than the linear distance between the two junctions 94. As a result, the support strut 93 may suppress a state where the proximal-side strut structures that have received the compressive force are twisted in the circumferential direction when the energy transfer elements are pressed against the tissue. Accordingly, in the medical device 10, the force of pressing the energy transfer elements against the tissue is less likely to be dispersed, whereby the energy transfer elements may be effectively pressed against the biological tissue.

Furthermore, the present disclosure also provides the method for forming a shunt. The method for forming a shunt is a method for forming a shunt including: inserting the medical device 10 described above from the inferior vena cava Iv into the right atrium HRa; inserting the expansion body 21 in the contracted state into a hole formed in the oval fossa; expanding the expansion body 21 in the hole to dispose a biological tissue surrounding the hole in the reception space 74 defined by the recess 55; sliding the pulling shaft 33 in the direction of the proximal end with respect to the shaft portion 31 to compress the expansion body 21 such that the distal-side upright portion 72 and the proximal-side upright portion 73 of the recess 55 approach each other; bringing the energy transfer elements disposed to face the recess 55 along the distal-side upright portion 72 or the proximal-side upright portion 73 of the recess 55 into contact with the biological tissue while relaxing the compressive force by deforming the buffer portion in a direction different from the direction from the force receiving portion 51 toward the distal-side top portion 57 along the distal-side expansion portion 56 or relaxing the compressive force by deforming the buffer portion disposed in the second expansion portion 54 in a direction different from the direction from the proximal end of the expansion body 21 toward the proximal-side top portion 59 along the proximal-side expansion portion 58; and cauterizing the biological tissue disposed in the reception space 74 using the energy transfer elements in contact with the biological tissue to inhibit occlusion due to natural healing of the hole.

According to the method for forming a shunt configured as described above, the compressive force is relaxed by deformation of the buffer portion in a direction different from the direction from the force receiving portion 51 toward the distal-side top portion 57 or in a direction different from the direction from the proximal end of the expansion body 21 toward the proximal-side top portion 59 when the compressive force is applied to the expansion body 21 by the pulling of the pulling shaft 33, whereby it becomes possible to suppress the state where the expansion body 21 is excessively expanded radially outward. Therefore, it becomes possible to suppress variations in the amount of expansion of the expansion body 21 toward the radial outside when the biological tissue is thick or thin. Thus, the method for forming a shunt is enabled to suppress variations in the amount of radial expansion of the expansion body 21, which is inserted into the hole penetrating the biological tissue and is expanded radially outward by the compressive force, due to variations in the thickness of the biological tissue. Therefore, the present method for forming a shunt is less likely to be affected by the variations in the thickness of the biological tissue, suppresses excessive expansion of the hole of the biological tissue, and enables highly safe and appropriate cauterization.

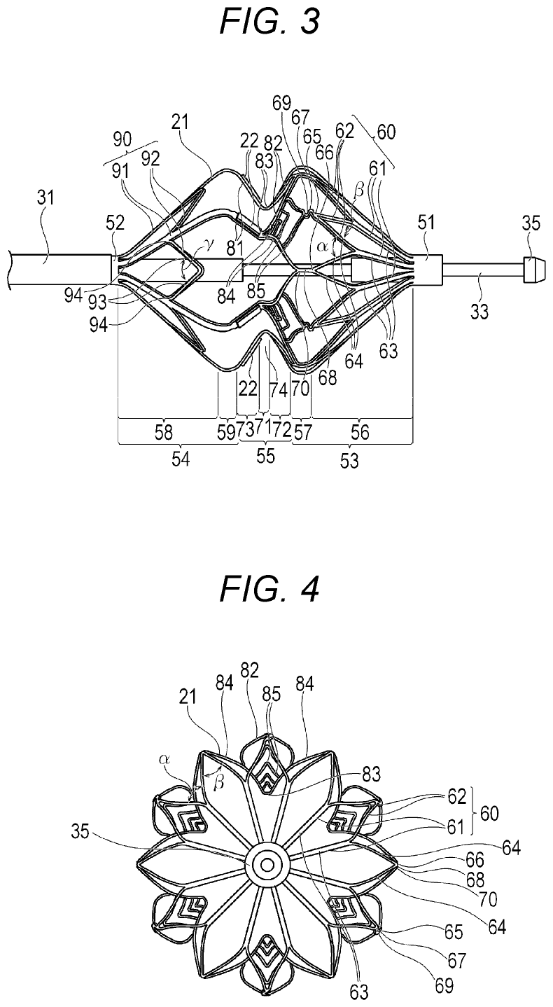

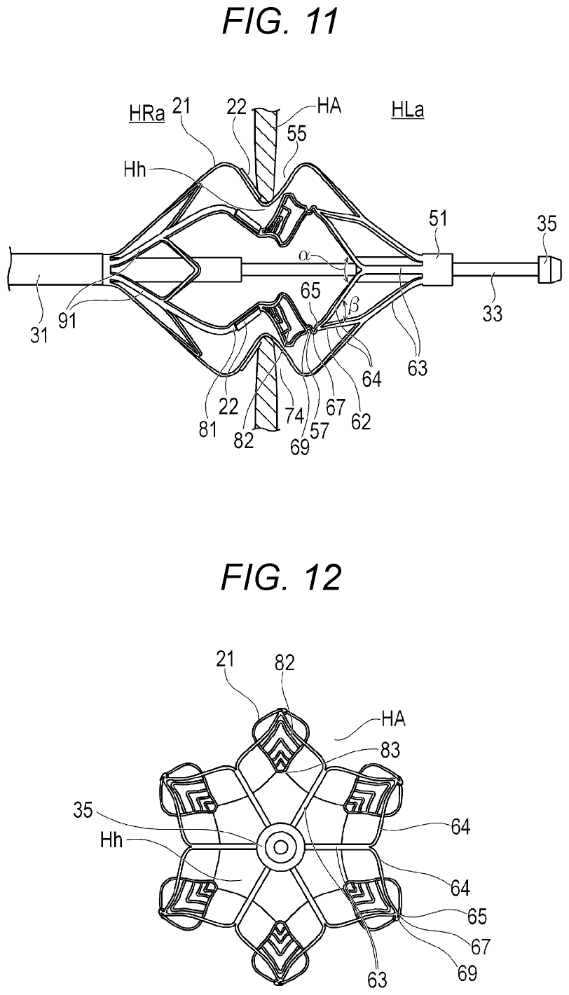

The present disclosure is not limited to the embodiment described above, and various modifications may be made by those skilled in the art within the technical idea of the present disclosure. Therefore, the position at which the buffer portion is disposed is not particularly limited as long as it is in the first expansion portion or the second expansion portion. For example, as in a first modified example illustrated in FIGS. 11 and 12, the number of the first struts 63 may be six, which is half, and the number of second struts 64 may be 12, which is half, as compared with the embodiment illustrated in FIGS. 1 to 4 described above. The first strut 63 is disposed in a phase different from that of the third strut 91, the energy transfer element arrangement portion 81, the facing portion 82, and the energy transfer element 22. In addition, the joint portions at which the second struts 64 gather together are all the first joint portions 65, and the second joint portion 66, the connecting strut 68, and the second distal-side top portion 70 illustrated in FIG. 3 may not be disposed. While the auxiliary curved portion 67 is disposed between the first joint portion 65 and the first distal-side top portion 69, the auxiliary curved portion 67 may not be disposed.

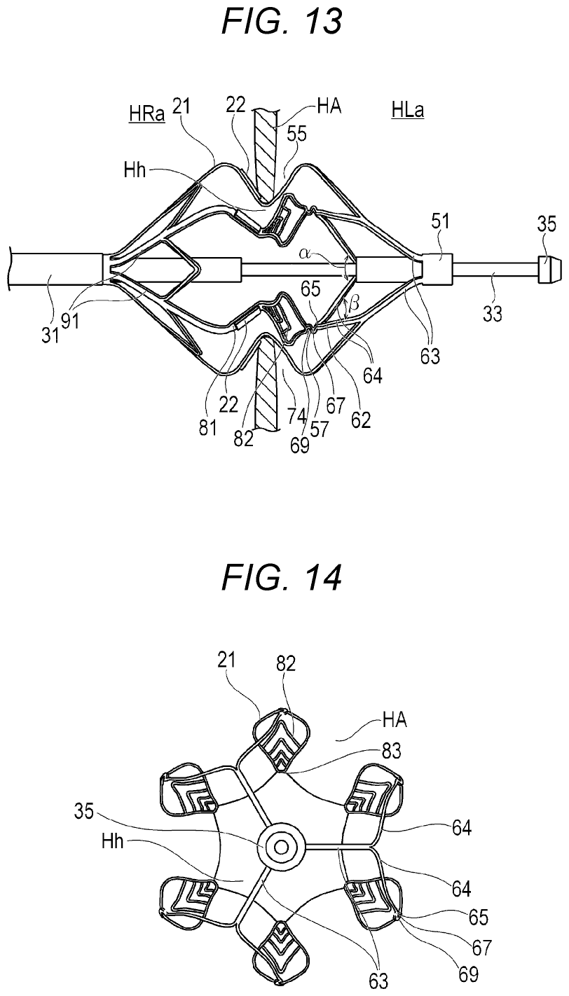

Furthermore, as in a second modified example illustrated in FIGS. 13 and 14, the number of the first struts 63 may be three, which is ¼, and the number of the second struts 64 (buffer portions) may be six, which is ¼, as compared with the embodiment illustrated in FIGS. 1 to 4. In addition, the six individual second struts 64 bifurcated from the three first struts 63 are coupled to the respective six auxiliary curved portions 67 (buffer portions) without being gathered together. The auxiliary curved portion 67 may not be disposed between the second strut 64 and the facing portion 82. The first strut 63 is disposed in a phase different from that of the third strut 91, the energy transfer element arrangement portion 81, the facing portion 82, and the energy transfer element 22.

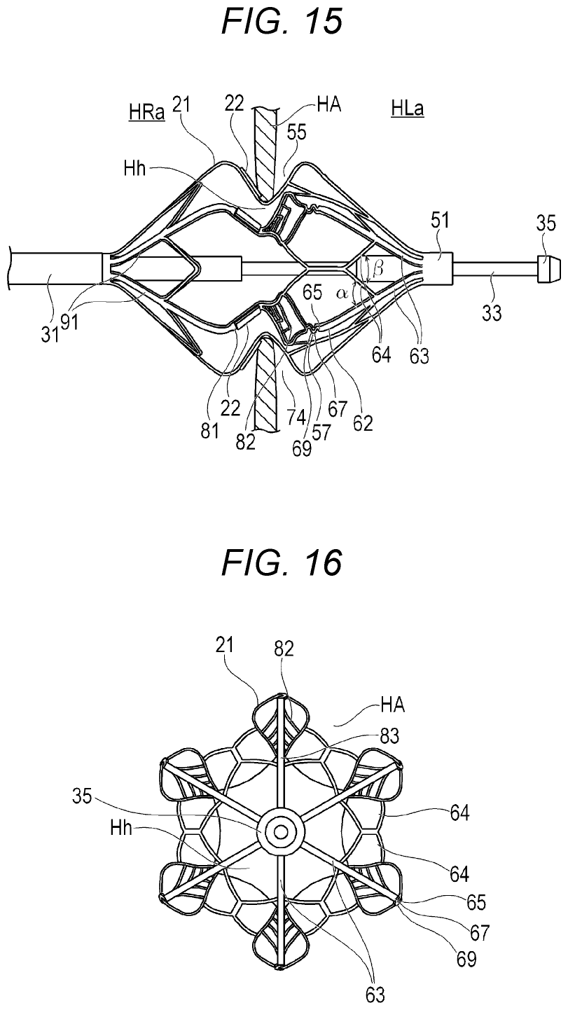

Furthermore, as in a third modified example illustrated in FIGS. 15 and 16, the number of the first struts 63 may be six, which is half, and the number of the second struts 64 (buffer portions) may be 12, which is half, as compared with the embodiment illustrated in FIGS. 1 to 4. The first strut 63 is disposed in the same phase as the third strut 91 and the energy transfer element 22, and is coupled to the force receiving portion 51 and the auxiliary curved portion 67 (buffer portion). Each of the second struts 64 branches from the middle in the length direction of the first strut 63. The joint portions at which the portions of the proximal sides of the second struts 64 are gathered together are all the second joint portions 66, and no first joint portion coupled to the auxiliary curved portion 67 is disposed. While the auxiliary curved portion 67 is disposed between the first strut 63 and the first distal-side top portion 69, the auxiliary curved portion 67 may not be disposed.

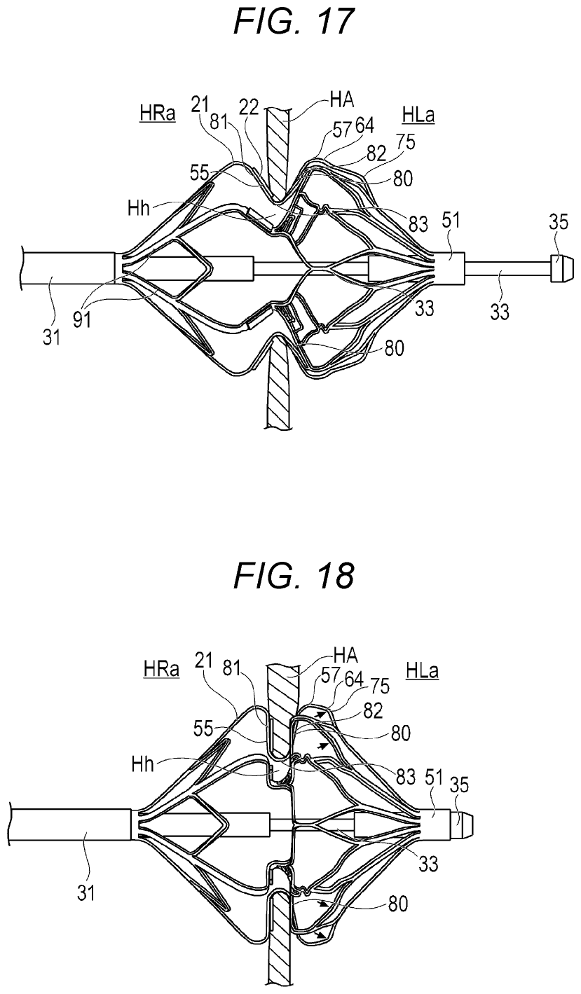

Furthermore, as in a fourth modified example illustrated in FIG. 17, each of the second struts 64 may have a bent portion 75 protruding radially outward in a natural state. The protruding direction is not limited to the radially outer side, and may be, for example, the radially inner side, the direction of the distal end, the direction of the proximal end, the circumferential direction, or the like. When the compressive force is applied to the expansion body 21, the bent portion 75 easily bends due to stress concentration, as illustrated in FIG. 18. The direction in which the bent portion 75 deforms is a direction different from the direction from the force receiving portion 51 toward the distal-side top portion 57 along the distal-side expansion portion 56. Therefore, the stress is concentrated on the bent portion 75 when the compressive force in the axial direction is applied to the expansion body 21, and the bent portion 75, which is a buffer portion, deforms in a direction (e.g., radial outside) different from the direction from the force receiving portion 51 toward the distal-side top portion 57 of the expansion body 21. As a result, the compressive force applied to the expansion body is relaxed, whereby the state where the expansion body is excessively expanded radially outward may be suppressed.

Furthermore, the second strut 64 may include a bent portion configured to, instead of protruding in the natural state as in the fourth modified example, easily deform in a direction different from the force receiving portion 51 toward the distal-side top portion 57 along the distal-side expansion portion 56 when the compressive force in the axial direction is applied to the expansion body 21. Such a bent portion may be set by making the width of the second strut 64 (circumferential length of the expansion body 21) smaller than the width of other struts of the expansion body 21, or making the thickness of the second strut 64 (radial length of the expansion body 21) smaller than the thickness of other struts of the expansion body 21.

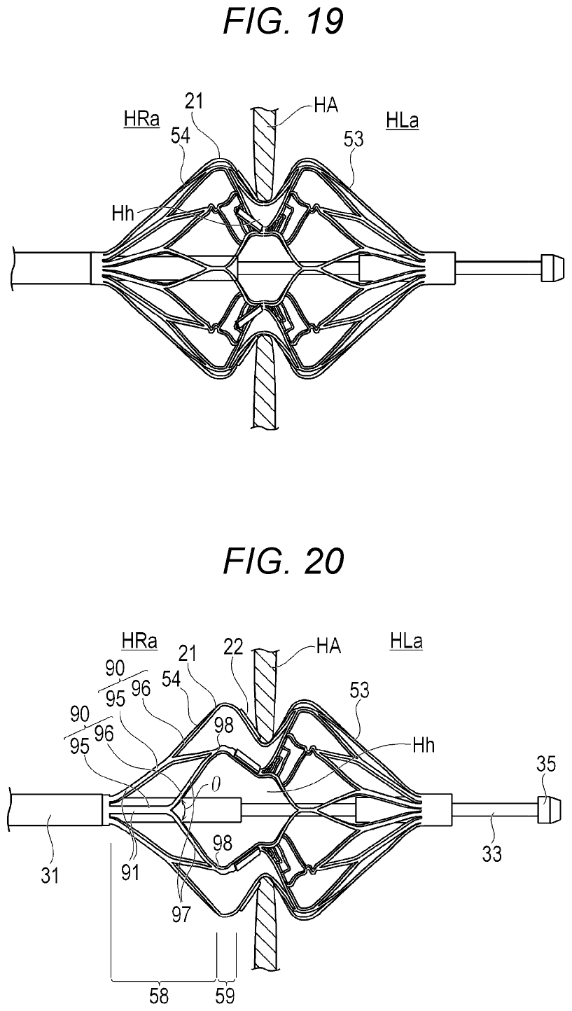

Furthermore, as in a fifth modified example illustrated in FIG. 19, the second expansion portion 54 on the proximal side may have a plane-symmetrical structure substantially similar to the first expansion portion 53 on the distal side.

Furthermore, as in a sixth modified example illustrated in FIG. 20, the second expansion portion 54 on the proximal side may have a branched structure. The second expansion portion 54 includes the plurality of proximal-side strut structures 90 extending radially outward from the distal end part of the shaft portion 31 toward the direction of the distal end and forming the proximal-side expansion portion 58. Each of the plurality of proximal-side strut structures 90 includes a third section 95 including the third strut 91 extending from the distal end part of the shaft portion 31 substantially parallel to the axial center of the expansion body 21 when viewed from the radial outside, and a fourth section 96 including two fourth struts 97 bifurcated from the distal end of the third section 95 substantially along the circumferential direction of the expansion body 21 and coupled to the proximal-side top portion 59. The fourth section 96 is bent such that a third bifurcation angle θ formed by the two bifurcated fourth struts 97 increases, thereby functioning as a buffer portion that relaxes the compressive force. The fourth section 96 of the proximal-side strut structure 90 is bent such that the third bifurcation angle θ formed by the two bifurcated fourth struts 97 increases, whereby the compressive force is relaxed and the state where the expansion body 21 is excessively expanded radially outward may be suppressed.

Furthermore, the fourth section 96 includes, in the vicinity of the proximal-side top portion 59, a plurality of third joint portions 98 in which each of the two fourth struts 97 joins one of the two fourth struts 97 of another fourth section 96 adjacent in the circumferential direction. As a result, the adjacent proximal-side strut structures 90 support each other in the circumferential direction, whereby the expansion body 21 is less likely to be twisted at the time of expansion. Therefore, an appropriate position of the biological tissue may be appropriately expanded by the expansion body 21, which enables appropriate cauterization.

Furthermore, when the through-hole Hh is sufficiently enlarged by the balloon 152 in the step of S1 of the method for forming a shunt described above, in the step of S4, the through-hole Hh may only be substantially gripped by the expansion body 21 instead of being enlarged by the expansion body 21.

Furthermore, the medical device 10 may be a device without the outer tube 30. In this case, a sheath corresponding to the outer tube 30 is separately prepared, and in the step of S2, the sheath is delivered to the vicinity of the atrial septum HA along the guide wire 11 in advance so that a distal end part of the sheath reaches the left atrium HLa via the through-hole Hh of the atrial septum HA. Next, the expansion body 21 of the medical device 10 is inserted into the sheath from the proximal end of the sheath, and the distal end part of the expansion body 21 is delivered to the left atrium HLa via the through-hole Hh of the atrial septum HA in a similar manner to FIG. 7.

Furthermore, the step of S4 of enlarging the diameter of the through-hole Hh using the expansion body 21 or gripping the through-hole Hh using the expansion body 21 and the step of S5 of confirming the hemodynamics in the vicinity of the through-hole Hh may be reversed. In this case, when the blood volume passing through the through-hole Hh does not reach the desired amount in the step of S5, the operator moves the outer tube 30 to the distal side to house the expansion body 21 in the outer tube 30, and then removes the expansion body 21 together with the outer tube 30 from the through-hole Hh. Next, the through-hole Hh is enlarged again using a balloon catheter including a balloon having an expansion diameter larger than that of the balloon 152 used in the step of S1, and the process returns to the step of S2.

The detailed description above describes embodiments of a medical device including an expansion body that expands in a living body and a method for forming a shunt. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device comprising:
an expansion body that includes a distal end part including a force receiving portion, the expansion body configured to be expandable and contractible in a radial direction;
an elongated shaft portion including a distal end part to which a proximal end of the expansion body is fixed;
a plurality of energy transfer elements disposed along the expansion body;
a pulling shaft that is disposed inside the shaft portion, the pulling shaft configured to be connectable to the force receiving portion of the expansion body by protruding from the distal end part of the shaft portion, and to be slidable with respect to the shaft portion; and
the expansion body including:
a first expansion portion including a distal-side expansion portion extending radially outward from the force receiving portion toward a direction of the proximal end and a distal-side top portion disposed on a proximal side of the distal-side expansion portion and convexly curved radially outward;
a second expansion portion including a proximal-side expansion portion extending radially outward from the distal end part of the shaft portion toward a direction of the distal end and a proximal-side top portion disposed on a distal side of the proximal-side expansion portion and convexly curved radially outward;
a recess that is recessed radially inward, extends to couple the proximal-side top portion with the distal-side top portion, and configured to define a reception space configured to receive a biological tissue when the expansion body is expanded;
the recess includes a bottom portion located on an innermost side in the radial direction, a distal-side upright portion extending radially outward from a distal end of the bottom portion to the distal-side top portion, and a proximal-side upright portion extending radially outward from a proximal end of the bottom portion to the proximal-side top portion;
one of the distal-side upright portion or the proximal-side upright portion includes a plurality of energy transfer element arrangement portions on which the plurality of individual energy transfer elements is disposed at a substantially regular interval in a circumferential direction of the expansion body;
another one of the distal-side upright portion or the proximal-side upright portion includes a plurality of facing portions facing the plurality of individual energy transfer elements when the expansion body is expanded;
the pulling shaft is configured to apply, to the expansion body via the force receiving portion, a compressive force configured to compress along an axial center of the shaft portion such that the plurality of energy transfer element arrangement portions and the plurality of facing portions approach each other by sliding in a direction of the proximal end with respect to the shaft portion; and
the expansion body includes a buffer portion that is disposed in the first expansion portion and is configured to relax the compressive force by deforming in a direction different from a direction from the force receiving portion toward the distal-side top portion along the distal-side expansion portion, or a buffer portion that is disposed in the second expansion portion and is configured to relax the compressive force by deforming in a direction different from a direction from the proximal end of the expansion body toward the proximal-side top portion along the proximal-side expansion portion.

2. The medical device according to claim 1, wherein
the first expansion portion includes a plurality of distal-side strut structures extending radially outward from the force receiving portion toward the direction of the proximal end and forming the distal-side expansion portion; and
each of the plurality of distal-side strut structures includes, as the buffer portion, a bent portion bendable in a direction different from a direction from the force receiving portion toward the distal-side top portion along each of the distal-side strut structures.

3. The medical device according to claim 2, wherein
each of the plurality of distal-side strut structures includes a first section that includes a first strut extending from the force receiving portion substantially parallel to the axial center of the expansion body when viewed from a radial outside, and a second section that includes two second struts bifurcated from a proximal end of the first section substantially along the circumferential direction of the expansion body and is coupled to the distal-side top portion; and
the second section is configured to function as the buffer portion that relaxes the compressive force by bending such that a bifurcation angle formed by the two bifurcated second struts increases.

4. The medical device according to claim 3, wherein the second section includes, in a vicinity of the distal-side top portion, a plurality of joint portions in which each of the two second struts joins one of the two second struts of another second section adjacent in the circumferential direction.

5. The medical device according to claim 4, wherein the second section includes an auxiliary curved portion configured to function as the buffer portion between the plurality of joint portions and the distal-side top portion disposed in a same phase as the energy transfer element arrangement portions or the facing portions in the circumferential direction of the expansion body.

6. The medical device according to claim 4, wherein
the plurality of distal-side strut structures includes the first sections and the joint portions twice as many as the plurality of energy transfer elements; and
the joint portions alternately include, in the circumferential direction of the expansion body, a first joint portion disposed in a same phase as the plurality of energy transfer element arrangement portions and the plurality of facing portions in the circumferential direction of the expansion body, and a second joint portion disposed in a phase different from the phase of the plurality of energy transfer element arrangement portions and the plurality of facing portions.

7. The medical device according to claim 6, further comprising:
an auxiliary curved portion configured to function as the buffer portion between the first joint portion and the distal-side top portion.

8. The medical device according to claim 3, wherein
the recess includes a recessed strut structure that is coupled to the distal-side strut structure via the distal-side top portion and defines the distal-side upright portion, the proximal-side upright portion, and the bottom portion; and
the recessed strut structure includes, in the bottom portion, a plurality of bottom connecting portions that couples individual pairs of the plurality of energy transfer element arrangement portions and the plurality of facing portions; and
the plurality of bottom connecting portions is disposed in a phase different from a phase of the first strut in the circumferential direction of the expansion body.

9. The medical device according to claim 1, wherein
the energy transfer element arrangement portions are disposed on the proximal-side upright portion; and
the buffer portion is disposed only on the distal-side expansion portion.

10. The medical device according to claim 9, wherein
the second expansion portion includes a plurality of proximal-side strut structures that extends radially outward from the distal end part of the shaft portion toward the direction of the distal end and forms the proximal-side expansion portion; and
each of the plurality of proximal-side strut structures includes a third strut that is disposed in a same phase as the plurality of energy transfer element arrangement portions in the circumferential direction of the expansion body and extends from the distal end part of the shaft portion to the proximal-side top portion substantially parallel to the axial center of the expansion body when viewed from a radial outside.

11. The medical device according to claim 10, wherein
the second expansion portion includes a plurality of secondary struts that couples the third struts adjacent in the circumferential direction in the plurality of proximal-side strut structures;
each of the plurality of secondary struts includes at least one support strut including two junctions joined to respective two third struts adjacent in the circumferential direction among a plurality of the third struts; and
each of a plurality of the support struts is formed to be longer than a linear distance between the two junctions.

12. The medical device according to claim 9, wherein
the second expansion portion includes a plurality of proximal-side strut structures that extends radially outward from the distal end part of the shaft portion toward the direction of the distal end and forms the proximal-side expansion portion;
each of the plurality of proximal-side strut structures includes a third section that includes a third strut extending from the distal end part of the shaft portion substantially parallel to the axial center of the expansion body when viewed from a radial outside, and a fourth section that includes two fourth struts bifurcated from a distal end of the third section substantially along the circumferential direction of the expansion body and is coupled to the proximal-side top portion; and
the fourth section is configured to function as the buffer portion that relaxes the compressive force by bending such that a bifurcation angle formed by the two bifurcated fourth struts increases.

13. The medical device according to claim 12, wherein the fourth section includes, in a vicinity of the proximal-side top portion, a plurality of third joint portions in which each of the two fourth struts joins one of the two fourth struts of another fourth section adjacent in the circumferential direction.

14. An expansion body configured to be expandable and contractible in a radial direction, the expansion body comprising:
a distal end part including a force receiving portion;
a first expansion portion including a distal-side expansion portion extending radially outward from the force receiving portion toward a direction of the proximal end and a distal-side top portion disposed on a proximal side of the distal-side expansion portion and convexly curved radially outward;
a second expansion portion including a proximal-side expansion portion extending radially outward from the distal end part of the shaft portion toward a direction of the distal end and a proximal-side top portion disposed on a distal side of the proximal-side expansion portion and convexly curved radially outward;
a recess that is recessed radially inward, extends to couple the proximal-side top portion with the distal-side top portion, and configured to define a reception space configured to receive a biological tissue when the expansion body is expanded;
the recess includes a bottom portion located on an innermost side in the radial direction, a distal-side upright portion extending radially outward from a distal end of the bottom portion to the distal-side top portion, and a proximal-side upright portion extending radially outward from a proximal end of the bottom portion to the proximal-side top portion;
one of the distal-side upright portion or the proximal-side upright portion includes a plurality of energy transfer element arrangement portions on which the plurality of individual energy transfer elements is disposed at a substantially regular interval in a circumferential direction of the expansion body;
another one of the distal-side upright portion or the proximal-side upright portion includes a plurality of facing portions facing the plurality of individual energy transfer elements when the expansion body is expanded; and
a buffer portion that is disposed in the first expansion portion and is configured to relax a compressive force by deforming in a direction different from a direction from the force receiving portion toward the distal-side top portion along the distal-side expansion portion, or a buffer portion that is disposed in the second expansion portion and is configured to relax the compressive force by deforming in a direction different from a direction from the proximal end of the expansion body toward the proximal-side top portion along the proximal-side expansion portion.

15. The expansion body according to claim 14, wherein
the first expansion portion includes a plurality of distal-side strut structures extending radially outward from the force receiving portion toward the direction of the proximal end and forming the distal-side expansion portion; and
each of the plurality of distal-side strut structures includes, as the buffer portion, a bent portion bendable in a direction different from a direction from the force receiving portion toward the distal-side top portion along each of the distal-side strut structures.

16. The expansion body according to claim 15, wherein
each of the plurality of distal-side strut structures includes a first section that includes a first strut extending from the force receiving portion substantially parallel to the axial center of the expansion body when viewed from a radial outside, and a second section that includes two second struts bifurcated from a proximal end of the first section substantially along the circumferential direction of the expansion body and is coupled to the distal-side top portion; and
the second section is configured to function as the buffer portion that relaxes the compressive force by bending such that a bifurcation angle formed by the two bifurcated second struts increases.

17. The expansion body according to claim 16, wherein the second section includes, in a vicinity of the distal-side top portion, a plurality of joint portions in which each of the two second struts joins one of the two second struts of another second section adjacent in the circumferential direction.

18. A method for forming a shunt according to the present disclosure forms, in an oval fossa, a shunt through which a right atrium communicates with a left atrium using a medical device including an expansion body that includes a distal end part including a force receiving portion, the expansion body being expandable and contractible in a radial direction, an elongated shaft portion including a distal end part to which a proximal end of the expansion body is fixed, a plurality of energy transfer elements disposed along the expansion body, and a pulling shaft that is disposed inside the shaft portion, connectable to the force receiving portion of the expansion body by protruding from the distal end part of the shaft portion, and slidable with respect to the shaft portion, in which the expansion body includes a first expansion portion including a distal-side expansion portion extending radially outward from the force receiving portion toward a direction of the proximal end and a distal-side top portion disposed on a proximal side of the distal-side expansion portion and convexly curved radially outward, a second expansion portion including a proximal-side expansion portion extending radially outward from the distal end part of the shaft portion toward a direction of the distal end and a proximal-side top portion disposed on a distal side of the proximal-side expansion portion and convexly curved radially outward, and a recess that is recessed radially inward, extends to couple the proximal-side top portion with the distal-side top portion, and defines a reception space that can receive a biological tissue when the expansion body is expanded, the recess includes a bottom portion located on an innermost side in the radial direction, a distal-side upright portion extending radially outward from a distal end of the bottom portion to the distal-side top portion, and a proximal-side upright portion extending radially outward from a proximal end of the bottom portion to the proximal-side top portion, one of the distal-side upright portion or the proximal-side upright portion includes a plurality of energy transfer element arrangement portions on which the plurality of individual energy transfer elements is disposed at a substantially regular interval in a circumferential direction of the expansion body, the other one of the distal-side upright portion or the proximal-side upright portion includes a plurality of facing portions facing the plurality of individual energy transfer elements when the expansion body is expanded, the pulling shaft is configured to apply, to the expansion body via the force receiving portion, a compressive force that makes compression along an axial center of the shaft portion such that the plurality of energy transfer element arrangement portions and the plurality of facing portions approach each other by sliding in a direction of the proximal end with respect to the shaft portion, and the expansion body includes a buffer portion that is disposed in the first expansion portion and is configured to relax the compressive force by deforming in a direction different from a direction from the force receiving portion toward the distal-side top portion along the distal-side expansion portion, or a buffer portion that is disposed in the second expansion portion and is configured to relax the compressive force by deforming in a direction different from a direction from the proximal end of the expansion body toward the proximal-side top portion along the proximal-side expansion portion, the method including:
inserting the medical device from an inferior vena cava into the right atrium;
inserting the expansion body in a contracted state into a hole formed in the oval fossa;
expanding the expansion body in the hole to dispose the biological tissue surrounding the hole in the reception space defined by the recess;
sliding the pulling shaft in the direction of the proximal end with respect to the shaft portion to compress the expansion body such that the distal-side upright portion and the proximal-side upright portion of the recess approach each other;
bringing the energy transfer elements disposed to face the recess along the distal-side upright portion or the proximal-side upright portion of the recess into contact with the biological tissue while relaxing the compressive force by deforming the buffer portion in a direction different from the direction from the force receiving portion toward the distal-side top portion along the distal-side expansion portion or relaxing the compressive force by deforming the buffer portion disposed in the second expansion portion in a direction different from the direction from the proximal end of the expansion body toward the proximal-side top portion along the proximal-side expansion portion; and
cauterizing the biological tissue disposed in the reception space using the energy transfer elements in contact with the biological tissue to inhibit occlusion due to natural healing of the hole.

19. The method according to claim 18, wherein
the first expansion portion includes a plurality of distal-side strut structures extending radially outward from the force receiving portion toward the direction of the proximal end and forming the distal-side expansion portion; and
each of the plurality of distal-side strut structures includes, as the buffer portion, a bent portion bendable in a direction different from a direction from the force receiving portion toward the distal-side top portion along each of the distal-side strut structures.

20. The method according to claim 19, wherein
each of the plurality of distal-side strut structures includes a first section that includes a first strut extending from the force receiving portion substantially parallel to the axial center of the expansion body when viewed from a radial outside, and a second section that includes two second struts bifurcated from a proximal end of the first section substantially along the circumferential direction of the expansion body and is coupled to the distal-side top portion; and
the second section functions as the buffer portion that relaxes the compressive force by bending such that a bifurcation angle formed by the two bifurcated second struts increases.
